(19) United States
(12) Patent Application Publication
(10) Pub. No.: US 2017/0224268 A1
(43) Pub. Date: Aug. 10, 2017

(54) SYSTEMS AND METHODS FOR DETECTING A LABOR CONDITION

(71) Applicants: Marco Altini, San Francisco, CA (US); Julien Penders, San Francisco, CA (US); Eric Dy, San Francisco, CA (US)

(72) Inventors: Marco Altini, San Francisco, CA (US); Julien Penders, San Francisco, CA (US); Eric Dy, San Francisco, CA (US)

(73) Assignee: Bloom Technologies NV, Genk (BE)

(21) Appl. No.: 15/429,215

(22) Filed: Feb. 10, 2017

Related U.S. Application Data
(60) Provisional application No. 62/293,714, filed on Feb. 10, 2016.

(51) Int. Cl. A61B 5/00; A61B 5/04; A61B 5/0408; A61B 5/0205; A61B 5/16; A61B 5/0492; A61B 5/0448; A61B 5/0488; A61B 5/11

(52) U.S. Cl. CPC A61B 5/4356; A61B 5/7267; A61B 5/7246; A61B 5/04882; A61B 5/04014; A61B 5/04085; A61B 5/1107; A61B 5/4362; A61B 5/1118; A61B 5/165; A61B 5/0492; A61B 5/0006; A61B 5/6833; A61B 5/6823; A61B 5/0448; A61B 5/0205; A61B 2560/0412; A61B 2560/0431; A61B 5/02405

(57) ABSTRACT

Systems and methods for monitoring the onset or occurrence of labor contractions and detecting or estimating labor in a pregnant female are provided.

### CROSS-REFERENCE TO RELATED APPLICATIONS

This application claims priority to U.S. Provisional Patent Application Ser. No. 62/293,714, entitled “Systems and Methods for Detecting a Labor Condition,” filed Feb. 10, 2016, the disclosure of which is hereby incorporated by reference in its entirety.

### TECHNICAL FIELD

This invention relates generally to the field of obstetrics and gynecology, and more specifically to new and useful systems and methods for detecting and characterizing labor.

### BACKGROUND

The process of childbirth or labor is largely performed through contractions of a woman's uterine muscle. Uterine contractions involve periodic tightening and relaxation of the uterine muscle. Pre-labor uterine contractions, including Braxton-Hicks contractions, may begin early in a pregnancy. These contractions are irregular and generally weak and do not result in delivery of a baby. Stronger, more regularly timed labor contractions result in tightening of the upper portion of a woman's uterus and relaxation and stretching of the cervix and lower portion of the uterus. Such changes facilitate delivery of the baby from the uterus through the cervix. A woman's progress through the childbirth process can be monitored based on the intensity, frequency, and duration of labor contractions.

In a healthcare setting, uterine contraction activity is commonly monitored using a tocograph or uterine pressure catheter. Such devices mechanically sense pressure changes caused by uterine contractions. The tocograph is strapped to a woman's midsection using a belt, and the pressure transducer is pressed against the woman's abdomen. The device is large and obtrusive and requires a woman to stay next to the bulky equipment, thus limiting her mobility once attached. Moreover, the device requires careful positioning in order to get a reliable measurement. As a consequence, the tocograph must be operated by a trained clinician. The uterine pressure catheter includes an intrauterine pressure sensor attached to a catheter; the device is inserted into a woman's uterus via the birth canal in order to detect changes in uterine pressure that occur during a contraction. Thus, the device is fairly intrusive and also must be operated by a trained clinician. Both tocographs and intrauterine pressure catheters measure the change in pressure that results from a contraction rather than the physiological phenomena leading to the contraction. As a result, their accuracy in characterizing contractions, especially the intensity of contractions, is not high.

The devices described above are only available in a healthcare setting and are typically used to estimate progression through the childbirth process once labor has begun. Pregnant women continue to face significant uncertainty outside of the healthcare setting when trying to determine whether contractions they experience are true labor contractions and whether it is an appropriate or necessary time to seek medical attention. The uncertainty pregnant women and their families face in deciphering whether a woman is, or soon will be, in labor causes significant anxiety and stress. The uncertainty may lead to over-utilization of the healthcare system due to false alarms. This may result in wasted time, wasted medical resources, and unnecessary medical costs. The uncertainty may alternatively cause women to wait too long to seek medical attention, resulting in unintentional deliveries outside of healthcare facilities. Delivering a child without a medical professional or birthing specialist present may increase the risk of complications to child and mother, eventually leading to increased risk of maternal and fetal death.

Accordingly, there is a need for new and useful systems and methods for detecting the onset or occurrence of labor contractions and, more generally, the onset or occurrence of labor. There is a need for systems and methods that detect and/or estimate labor in pregnant women.

### SUMMARY

Various aspects of the present disclosure are directed to systems, devices, and methods that address one or more of the needs identified above.

One aspect of the disclosure is directed to a computer-implemented method for identifying a labor state in a pregnant female. In various embodiments, the method includes: acquiring a physiological signal from a physiological sensor; processing the physiological signal to identify and extract a parameter of interest from the physiological signal; and analyzing the parameter of interest to determine whether the parameter is indicative of a labor state.

In some embodiments, the method further includes developing a personalized parameter baseline. In some such embodiments, analyzing the parameter of interest to determine whether the parameter is indicative of a labor state includes: comparing the parameter of interest to the personalized parameter baseline to identify a deviation from the personalized parameter baseline, and determining whether the deviation is indicative of the labor state. The parameter of interest may be tracked over time to develop the personalized parameter baseline.

In some embodiments, a plurality of parameters of interest are identified and extracted from the physiological signal. In some such embodiments, analyzing the parameter of interest to determine whether the parameter is indicative of a labor state includes: identifying a pattern in the plurality of parameters, and determining whether the pattern is indicative of the labor state. The plurality of parameters may include physiological and behavioral parameters.

In some embodiments, analyzing the parameter of interest to determine whether the parameter is indicative of a labor state includes feeding the parameter into a machine learning model trained to detect labor. The machine learning model may include one or more of: a generalized linear model, a decision tree, a support vector machine, a k-nearest neighbor, a neural network, a deep neural network, a random forest, and a hierarchical model.

In some embodiments, analyzing the parameter of interest to determine whether the parameter is indicative of the labor state includes comparing the parameter to community data stored in a database. The community data may include one or more of: recorded trends, rules, correlations, and observations generated from tracking, aggregating, and analyzing parameters from a plurality of users.

Acquiring a physiological signal may include acquiring a plurality of physiological signals from a plurality of physiological sensors. In some embodiments, acquiring a physiological signal includes acquiring one or more of: an electrohysterography signal and a signal indicative of maternal uterine activity, maternal uterine muscle contractions, maternal heart electrical activity, maternal heart rate, fetal movement, fetal heart rate, maternal activity, maternal stress, and fetal stress.

In some embodiments, processing the physiological signal to identify and extract a parameter of interest includes identifying and extracting one or more of: a maternal heart rate metric, a maternal heart rate variability metric, a fetal heart rate metric, a fetal heart rate variability metric, a range of an electrohysterography signal, a power of an electrohysterography signal in a specific frequency band, a frequency feature of an electrohysterography signal, a time-frequency feature of an electrohysterography signal, a frequency of contractions, a duration of contractions, and an amplitude of contractions.

In some embodiments, the method further includes generating an alert related to the labor status. In some embodiments, the method further includes sharing the labor status or an alert related to the labor status with a contact. In some embodiments, the method further includes transmitting the labor status or an alert related to the labor status with a healthcare provider or labor support professional. In some embodiments, the method further includes performing an action based on the labor status. For example, in some embodiments, the method includes contacting a service provider to request services if the labor status is positive.

In some embodiments, the method further includes determining a probability that the pregnant female is experiencing labor-inducing contractions. A degree of certainty around the determined probability may also be determined. Additionally or alternatively, the method may further include determining a probability that the pregnant female will enter the labor state within a given time period. Additionally or alternatively, the method may further include determining an estimate of time until the pregnant female enters the labor state.

Another aspect of the disclosure is directed to a system for identifying a labor state in a pregnant female. In various embodiments, the system includes a physiological sensor, a processor communicatively coupled to the physiological sensor, and a computer-readable medium having non-transitory, processor-executable instructions stored thereon. Execution of the instructions causes the processor to perform any one or more of the methods described above or elsewhere herein.

In some embodiments of the system, the physiological sensor includes at least one measurement electrode and at least one reference electrode. The system may include one or a plurality of physiological sensors. In some embodiments, acquiring a physiological signal includes acquiring a plurality of physiological signals. The physiological sensor may include one or more physiological sensors configured, for example, to measure one or more of an electrohysterography signal, maternal uterine activity, maternal uterine muscle contractions, maternal heart electrical activity, maternal heart rate, fetal movement, fetal heart rate, maternal activity, maternal stress, and fetal stress. The one or more physiological sensors may sense one or more biopotential signals. In some embodiments, the parameter of interest includes one or more of: a maternal heart rate metric, a maternal heart rate variability metric, a fetal heart rate metric, a fetal heart rate variability metric, a range of an electrohysterography signal, a power of an electrohysterography signal in a specific frequency band, a frequency feature of an electrohysterography signal, a time-frequency feature of an electrohysterography signal, a frequency of contractions, a duration of contractions, a variability in contractions, and an amplitude of contractions.

In some embodiments, the system also includes a portable and wearable sensor module. The sensor module includes the physiological sensor, an electronic circuit, and a wireless antenna. In some such embodiments, the sensor module further includes the processor and the computer-readable medium. Such a sensor module may be in wireless communication with a mobile computing device. In other embodiments, the processor and the computer-readable medium are located within a mobile computing device, and the sensor module is in wireless communication with the mobile computing device.

In some embodiments having a mobile computing device, the mobile computing device is a smartphone, a smart watch, smart glasses, smart contact lenses, other wearable computer, a tablet, a laptop, or a personal computer.

In some embodiments having a wearable sensor module, the sensor module connects to or forms a portion of: a patch, a belt, a strap, a band, a t-shirt, the elastic of a pair of pants, or other clothing or other wearable accessory.

These and other aspects of the disclosure are illustrated in the figures and described in more detail below.

### BRIEF DESCRIPTION OF THE DRAWINGS

FIG. 1 depicts a block diagram of one embodiment of a system for identifying a labor state in a pregnant female.

FIG. 2 depicts a block diagram of another embodiment of a system for identifying a labor state in a pregnant female.

FIG. 3 depicts a block diagram of another embodiment of a system for identifying a labor state in a pregnant female.

FIG. 4 depicts a top view of one embodiment of a sensor module, which forms a portion of a system for identifying a labor state in a pregnant female.

FIG. 5 depicts a top view of another embodiment of a sensor module, which forms a portion of a system for identifying a labor state in a pregnant female.

FIG. 6 depicts a perspective view of one embodiment of a sensor module being applied to the abdominal region of a pregnant woman.

FIG. 7 depicts a perspective view of another embodiment of a sensor module being applied to the abdominal region of a pregnant woman.

FIG. 8 depicts a perspective view of another embodiment of a sensor module being applied to the abdominal region of a pregnant woman.

FIG. 9 depicts a flow chart of one embodiment of a method for identifying a labor state in a pregnant female.

FIG. 10 depicts a flow chart of another embodiment of a method for identifying a labor state in a pregnant female.

FIG. 11 depicts a flow chart of another embodiment of a method for identifying a labor state in a pregnant female.

### DETAILED DESCRIPTION

The foregoing is a summary, and thus, necessarily limited in detail. The above mentioned aspects, as well as other aspects, features, and advantages of the present technology will now be described in connection with various embodiments. The inclusion of the following embodiments is not intended to limit the invention to these embodiments, but rather to enable any person skilled in the art to make and use this invention. Other embodiments may be utilized and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Disclosed herein are systems and methods for monitoring the onset or occurrence of labor contractions and detecting or estimating labor in a pregnant female.

Labor is associated with uterine contractions, and each contraction originates with the electrical activation of uterine cells, similar to the activation of muscle cells. Electrohysterography (EHG) is the measure of uterine electrical activity, and compared to pressure monitoring methods, it is a more direct, and thus, more accurate and reliable means of monitoring contractions. By acquiring an EHG signal and extracting and analyzing physiological parameters from the signal, it becomes possible to determine if a woman is, or soon will be, in labor. Thus, various systems and methods provided herein depend, at least in part, on the detection, characterization, and analysis of EHG signals. In various embodiments, a portable EHG monitoring device or system is used, such as any of the devices or systems described in PCT/US2015/058153 to Bloom Technologies NV, filed on Oct. 29, 2015 and entitled “A Method and Device for Contraction Monitoring,” the disclosure of which is herein incorporated by reference in its entirety.

While other devices have been developed to detect EHG signals, past devices are not configured to predict or detect the onset of labor. For example, US Publ. No. 2012/0150010 to Hayes-Gill et al. and US Publ. No. 2007/0255184 to Shennib describe devices and methods for monitoring uterine activity based on EHG. However, such devices are limited in their functionality. These devices merely provide a measurement of the contraction signal and do not perform any further analysis on the signal. As a result, they are of limited value to pregnant women outside of a healthcare facility, requiring the intervention of an experienced clinician to interpret the results. Accordingly, a need exists for systems and methods that can be used by a pregnant woman in any environment to determine the status of a pregnancy. In particular, a need exists for systems and methods that can monitor and analyze contractions and other physiological signs to determine whether a woman is, or soon will be, in labor. At least some of the systems and methods disclosed herein fill this need.

In general, the systems and methods described herein include a sensor module used to monitor pregnancy or labor in a pregnant woman (i.e., a pregnant female human) or other pregnant female animal. Results of the monitoring may be provided to the pregnant woman being monitored and/or to a gynecologist, obstetrician, other physician, nurse practitioner, veterinarian, other healthcare provider, doula, midwife, other birthing specialist, spouse, partner, parent, sibling, other family member, friend, a healthcare facility administrator, a service provider who may provide ride-sharing, taxi, childcare, or other services to a woman in labor, or any other individual with whom the pregnant woman wishes to share such information.

As used herein, “pregnant woman” and “pregnant female” may be used interchangeably. It will be appreciated by one skilled in the art that each of the embodiments described herein may be used to monitor and detect a labor status in any pregnant mammal regardless of species.

As used herein, a “labor status” refers to a determination regarding the state of being in labor. Labor, or childbirth, is a process having various stages. In the first stage of labor (i.e., dilation), contractions become increasingly regular, the cervix dilates, and the baby descends to the mid-pelvis. In the second stage of labor (i.e., expulsion), the baby progresses through the birth canal (i.e., the cervix and vagina) and is expelled from the mother's body. The third stage of labor (i.e., placental stage) involves the delivery of the placenta and fetal membranes. The labor status may be positive (i.e., labor has begun) or negative (i.e., labor has not yet begun). The labor status may include a prediction of time until labor or a likelihood of beginning labor within a specified time period. The labor status may include a degree of likelihood that a woman is, or soon will be, in labor.

### System

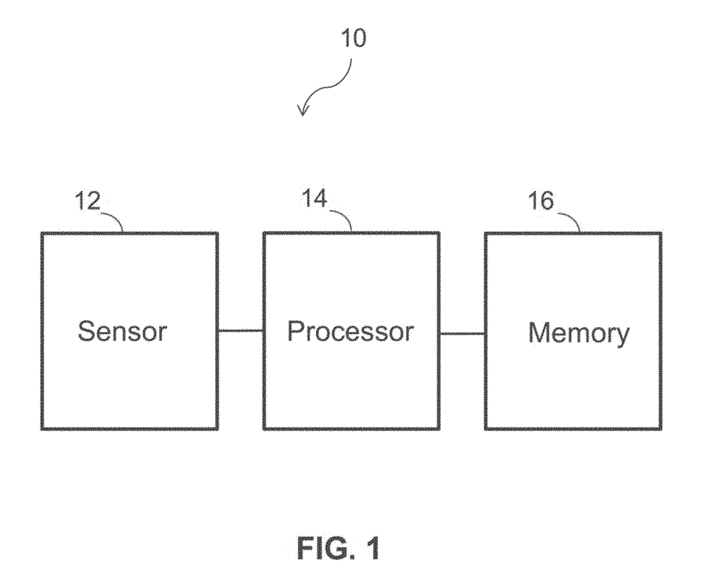

As shown in FIG. 1, in various embodiments, a system 10 for determining a labor status of a woman includes at least a physiological sensor 12 in electrical communication with a processor 14 and a computer-readable medium (i.e., memory) 16. FIG. 1 illustrates a functional block diagram, and it is to be appreciated that the various functional blocks of the depicted system 10 need not be separate structural elements. For example, in some embodiments, the processor 14 and memory 16 may be embodied in a single chip or two or more chips.

The physiological sensor 12 includes at least one measurement electrode and at least one reference electrode. In some configurations, one reference electrode and a plurality of measurement electrodes are present in the sensor 12. The system 10 may include one or a plurality of physiological sensors 12. For example, the physiological sensor 12 may include one or more sensors configured to measure an electrohysterography (EHG) signal, maternal uterine activity, maternal uterine muscle contractions, maternal heart electrical activity, maternal heart rate, fetal movement, fetal heart rate, maternal activity, maternal stress, and/or fetal stress. The one or more physiological sensors 12 may sense one or more biopotential signals. In one non-limiting embodiment, the physiological sensor 12 includes an EHG sensor and an electrocardiogram (ECG) sensor.

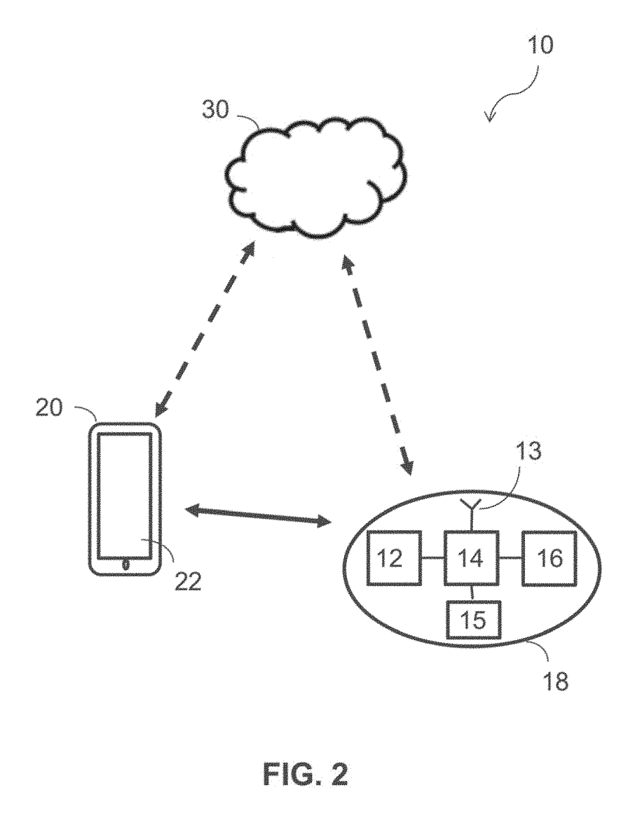

The physiological sensor 12 of various embodiments is configured for placement on an outer surface of a woman's body. In some embodiments, the sensor 12 is reusable; in other embodiments, the sensor 12 is disposable. In at least some embodiments, the sensor 12 is configured for placement over the belly or abdominal region of a pregnant woman. In some embodiments, the sensor 12 forms a portion of a sensor module. Various sensor module embodiments are described in more detail below with reference to FIGS. 2-8.

The processor 14 of FIG. 1 may be a general purpose microprocessor, a digital signal processor (DSP), a field programmable gate array (FPGA), an application specific integrated circuit (ASIC), or other programmable logic device, or other discrete computer-executable components designed to perform the functions described herein. The processor may also be formed of a combination of computing devices, for example, a DSP and a microprocessor, a plurality of microprocessors, one or more microprocessors in conjunction with a DSP core, or any other suitable configuration.

In some embodiments, the processor 14 is coupled, via one or more buses, to the memory 16 in order to read information from, and optionally write information to, the memory 16. The memory 16 may be any suitable computer-readable medium that stores computer-readable instructions for execution by a processor 14. For example, the computer-readable medium may include one or more of RAM, ROM, flash memory, EEPROM, a hard disk drive, a solid state drive, or any other suitable device. In some embodiments, the computer-readable instructions include software stored in a non-transitory format. The software may be programmed into the memory 16 or downloaded as an application onto the memory 16. The software may include instructions for running an operating system and/or one or more programs or applications. When executed by the processor 14, the programs or applications may cause the processor 14 to perform a method of detecting or estimating labor in a pregnant female. Some such methods are described in more detail elsewhere herein.

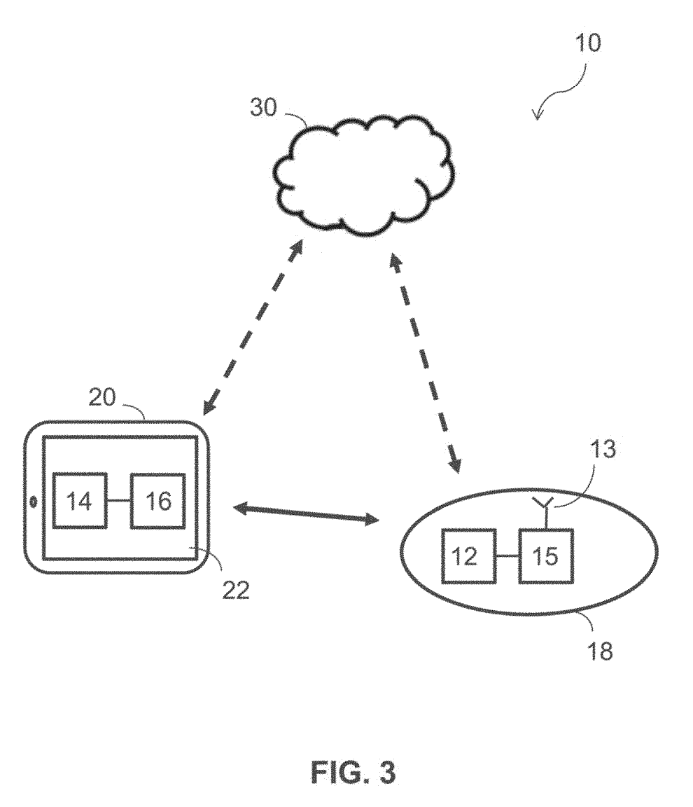

As shown in FIGS. 2 and 3, the system 10 may further include a sensor module 18 and a mobile computing device 20. In some embodiments, the system 10 also includes a server 30. In some embodiments, such as the embodiment of FIG. 2, the sensor 12, processor 14, and memory 16 are each positioned on or in the sensor module 18. An electronic circuit 15 and wireless antenna 13 may also be provided on or in the sensor module 18. In such embodiments, physiological signals are: sensed by the sensor 12; amplified, filtered, digitized and/or otherwise processed by the electronic circuit 15; and analyzed by the processor 14. Execution of instructions stored in memory 16 causes the processor 14 on the sensor module 18 to perform one or more of the methods of detecting a labor status described elsewhere herein. Analyzed data may be transmitted via the antenna 13 to one or both of the mobile computing device 20 and the server 30 for visual or audio presentation to a user, additional analysis, and/or storage.

In other embodiments, such as the embodiment of FIG. 3, the sensor 12 is positioned on or in the sensor module 18 with the electronic circuit 15 and wireless antenna 13, while a mobile computing device 20 houses the processor 14 that performs a method of detecting the labor status of a pregnant female and the memory 16 that stores instructions for performing the method. In such embodiments, physiological signals are sensed by the sensor 12 and amplified, filtered, digitized and/or otherwise processed by the electronic circuit 15, and the processed signals are transmitted via the antenna 13 to the mobile computing device 20. The processor 14 of the mobile computing device 20 analyzes the processed signals and detects a labor status, as described elsewhere herein. The analyzed data may be saved, shared with contacts, or presented to a user via the mobile computing device 20. In some such embodiments, some of or all the analyzed data may be transmitted from the mobile computing device 20 to a server 30 for storage.

In some embodiments, the electronic circuit 15 includes an operational amplifier, a low-pass, high-pass, or band-pass filter, an analog-to-digital (AD) converter, and/or other signal processing circuit components configured to amplify, filter, digitize, and/or otherwise process the physiological signal. The electronic circuit 15 may additionally include a power supply or power storage device, such as a battery or capacitor to provide power to the other electronic components. For example, the electronic circuit 15 may include a rechargeable (e.g., lithium ion) or disposable (e.g., alkaline) battery.

In some embodiments, the antenna 13 includes one or both of a receiver and a transmitter. The receiver receives and demodulates data received over a communication network. The transmitter prepares data according to one or more network standards and transmits data over a communication network. In some embodiments, a transceiver antenna 13 acts as both a receiver and a transmitter for bi-directional wireless communication. As an addition or alternative to the antenna 13, in some embodiments, a databus is provided within the sensor module 18 so that data can be sent from, or received by, the sensor module 18 via a wired connection.

In some embodiments, there is one-way or two-way communication between the sensor module 18 and the mobile computing device 20, the sensor module 18 and the server 30, and/or the mobile computing device 20 and the server 30. The sensor module 18, mobile computing device 20, and/or server 30 may communicate wirelessly using Bluetooth, low energy Bluetooth, near-field communication, infrared, WLAN, Wi-Fi, CDMA, LTE, other cellular protocol, other radiofrequency, or another wireless protocol. Additionally or alternatively, sending or transmitting information between the sensor module 18, the mobile computing device 20, and the server 30 may occur via a wired connection such as IEEE 1394, Thunderbolt, Lightning, DVI, HDMI, Serial, Universal Serial Bus, Parallel, Ethernet, Coaxial, VGA, or PS/2.

In some embodiments, the mobile computing device 20 is a computational device wrapped in a chassis that includes a visual display with or without touch responsive capabilities (e.g., Thin Film Transistor liquid crystal display (LCD), in-place switching LCD, resistive touchscreen LCD, capacitive touchscreen LCD, organic light emitting diode (LED), Active-Matrix organic LED (AMOLED), Super AMOLED, Retina display, Haptic/Tactile touchscreen, or Gorilla Glass), an audio output (e.g., speakers), a central processing unit (e.g., processor or microprocessor), internal storage (e.g., flash drive), n number of components (e.g., specialized chips and/or sensors), and n number of radios (e.g., WLAN, LTE, WiFi, Bluetooth, GPS, etc.). In some embodiments, the mobile computing device 20 is a mobile phone, smartphone, smart watch, smart glasses, smart contact lenses, or other wearable computing device, tablet, laptop, netbook, notebook, or any other type of mobile computing device. In some embodiments, the mobile computing device 20 may be a personal computer.

In some embodiments, the server 30 is a database server, application server, internet server, or other remote server. In some embodiments, the server 30 may store user profile data, historical user data, historical community data, algorithms, machine learning models, software updates, or other data. The server 30 may share this data with the mobile computing device 20 or the sensor module 18, and the server 30 may receive newly acquired user data from the sensor module 18 and/or the mobile computing device 20.

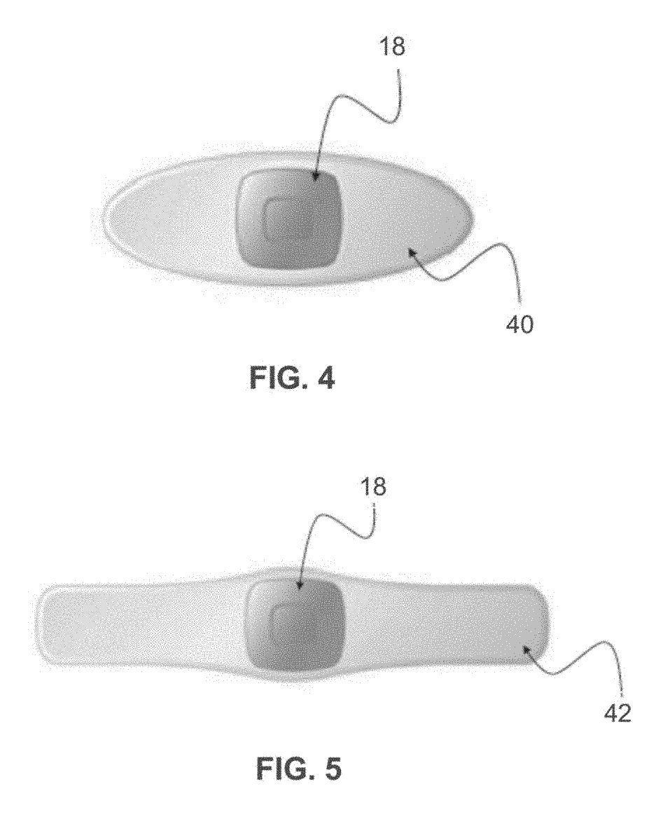

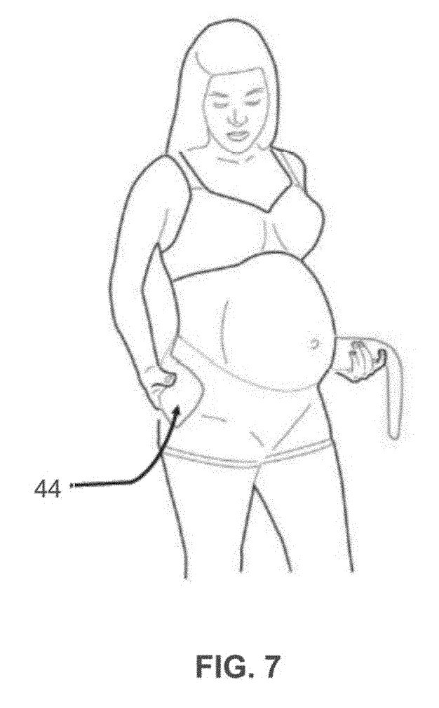

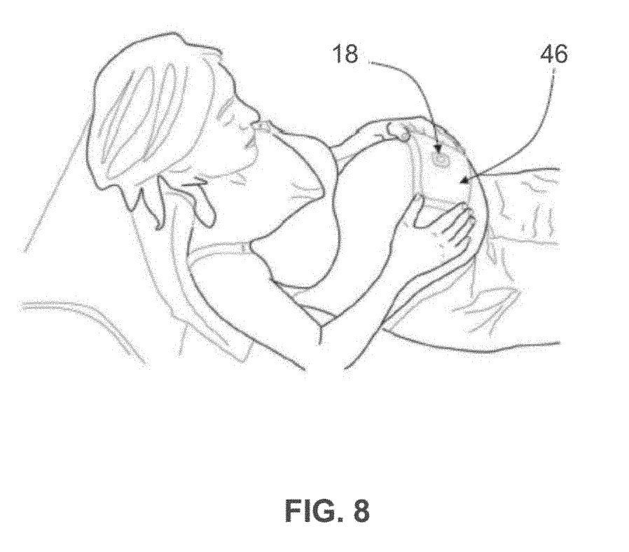

A few non-limiting examples of sensor modules 18 are depicted in FIGS. 4-8. By comparing the sensor modules of FIGS. 4-8, one can easily understand that the sensor module 18 can take many different form factors. The sensor module 18 of various embodiments has many different shapes, sizes, colors, materials, and levels of conformability to the body. The sensor module 18 may connect to, be embedded within, or form a portion of: a patch 40, 42 (e.g., FIGS. 4-6), a strap, belt, or band 44 (e.g., FIG. 7), or a blanket/cover 46 (e.g., FIG. 8), t-shirt, pants, underwear, or other article of clothing or wearable accessory. More details about each of these and other sensor module configurations is provided in PCT/US2015/058153 to Bloom Technologies NV, the disclosure of which is herein incorporated by reference in its entirety.

Turning to FIG. 4, the device for contraction monitoring comprises an electrode patch 40 and a sensor module 18, advantageously combined to monitor at least one channel of uterine contraction signals. The electrode patch 40 and the sensor module 18 may be in one part or may be made of two separate parts. The two separate parts can be provided with a mechanical and electrical system for attaching one to the other, such as a clipping system, a magnet. Other embodiments are described in the description.

FIG. 5 illustrates another embodiment of the device for contraction monitoring. By comparing FIG. 4 and FIG. 5, one will easily understand that the electrode patch 40, 42 or the sensor module 18 can take many different form factors.

Stated somewhat differently, the device for contraction monitoring can take many different shape, size, color, material and level of conformability to the body. The device may or may not take the form of a plaster. For example, the device may be integrated in a piece of garment. Or the device may take the form of a piece of clothing or textile. Or the device may take the form of a belt that is worn around the abdomen. For the last three examples, the electrode patch 40, 42 may be an integral part of the piece of garment, clothing or belt, or may be attached to such piece of garment, clothing or belt.

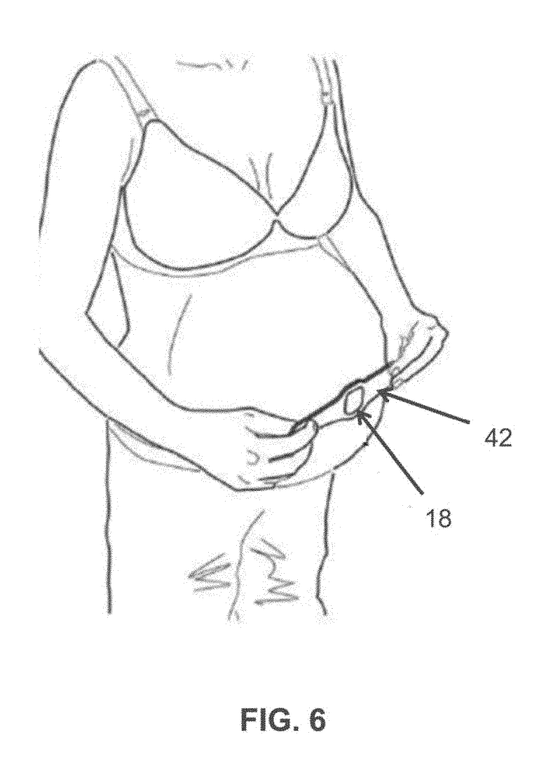

FIG. 6 shows an exemplary embodiment of the contraction monitoring device, wherein the electrode patch 42 and the sensor module 18 can be integrated and encapsulated into one unique part solely making the device. Preferably, the contraction monitoring device of FIG. 6 can have at least three electrodes, including one measurement electrode located on one extremity of the device, one reference electrode located on the other extremity of the device, and one bias electrode in the middle. Such configuration enables the measurement of one channel bio-potential signal, along the horizontal direction. In some embodiments, the device of FIG. 6 can have 4 electrodes, two measurement electrodes located on the two extremities, one reference electrode located in the middle of the device, and one bias electrode located between a measurement electrode and the reference electrode. Advantageously, a variant of the device of FIG. 6 (not shown) can have 5 electrodes, two measurement electrodes located on the two extremities of the device, one reference electrode located in the middle of the device, one additional measurement electrode located below the reference electrode, at 90 degrees from the line between the first three electrodes, and one bias electrode located between a measurement electrode and the reference electrode. Such configuration enables the measurement of two channels bio-potential signals, one along the horizontal direction and one along the vertical direction. In a further embodiment, the device can be attached to the body using an adhesive layer. In another embodiment, the adhesive layer can be replaced by the user. In another exemplary embodiment the device can be attached to the body using a strap or a piece of textile that can maintain the device in contact with the body.

FIG. 7 shows an exemplary embodiment of the contraction monitoring device 44, wherein the electrode patch and the sensor module can be integrated in a textile or clothing accessory. Examples of clothing accessory can include but are not limited to a shirt, T-shirt, belly-band, a pregnancy support belt or a belt. In some embodiments, the contraction monitoring device may have at least three electrodes arranged next to each other so that one measurement electrode is located on the right (respectively left) side of the abdomen, one reference electrode is located on the left (respectively right) side of the abdomen, and one bias electrode in the middle. In some embodiments, the device of FIG. 7 can have a fourth electrode positioned at 90 degrees from the linear arrangement, in the center of the abdomen. This fourth electrode can provide a measurement of the bio-potential signals in the vertical direction. In some embodiments, the device of FIG. 7 can have a fifth electrode positioned at the back of the woman, and providing a signal free of uterine activity but carrying physiological and recording artifacts, that can be used in processing the bio-potential signals to obtain cleaner and more accurate EHG, maternal ECG (mECG), and fetus ECG (fECG) signals.

FIG. 8 shows one embodiment of the contraction monitoring device, wherein the electrode patch 46 and the sensor module 18 can be integrated in an accessory of everyday life that can be 18 can be integrated in a pillow or in a cover.

As it can be seen from FIGS. 4-8, the device for contraction monitoring is integrated in a small and easy to use form factor that does not require to be operated by clinical staff. Stated somewhat differently, the device for contraction monitoring is advantageously implemented in such a way that a pregnant woman can operate it on her own. The small size and extreme miniaturization can be achieved thanks low-power electronics system design, that is a combination of low-power circuit design, low-power architecture design and firmware optimization. Low-power system design allows minimizing the size of the battery and therefore can achieve very small size for the overall system. The ease of use can come from a combination of smart electronics and high level of integration. With smart electronics, the device can automatically turn on when it is positioned on the body, or the device can automatically detect contractions and trigger feedback accordingly, or the system can automatically detect a specific situation—for example the fact that the woman is moving—and adapt its signal processing accordingly. With high level of integration, the electrode patch can integrate all wires to the electrode, and provide a very simple way for the user to connect the sensor to the electrode patch. Connecting the electrode patch to the sensor module can be done through magnetic interface, through a snap on mechanism, through a slide on mechanism, through a screw on mechanism, or any other mechanisms that provide a good mechanical and electrical contact between the sensor module and the electrode patch.

The use of an electrode patch improves the reliability of contraction monitoring as it is not possible for a user to misplace the different electrodes relatively to each other, as they are always in the same relative position. The use of an electrode patch improves the experience and the ease of use of contraction monitoring as it does not require attaching multiple electrodes to the abdomen, but only requires to attach one single electrode patch.

The device can be designed such that it is clear for the pregnant woman how to wear the device, and where to place it. The device can be designed such that it is very easy to put on. Preferably, the pregnant woman simply has to take the sensor module, attach it to the electrode patch, and wear it.

In some embodiments, the electrode patch comprises at least two electrodes, referred to as the measurement electrode and the reference electrode, and allowing the measurement of one channel bio-potential signal. In an alternative embodiment of the device, the electrode patch can include a third electrode, which can be used for biasing the signal acquisition electronics to the body voltage, or for applying a common mode voltage to the body in order to reduce the measurement noise, a measurement principle also known as right leg drive. In another alternative embodiment of the device, the electrode patch can include additional measurement electrodes, allowing the measurement of multiple channels of bio-potential signals, leading to multiple channels of uterine contraction signals. The multiple measurement electrodes can be positioned on different locations on the abdomen, advantageously providing multi-dimensional measurement of the uterine electrical activity. The electrodes may or may not include conductive gel. Conductive gel may be used to improve the quality of the contact between the body and the electrodes. The electrode patch may or may not be adhesive.

### Methods

Some of or all the above-described components or additional or alternate components may function to detect or estimate labor in a pregnant female. Some of the methods employed to detect or estimate labor in pregnant females are described below.

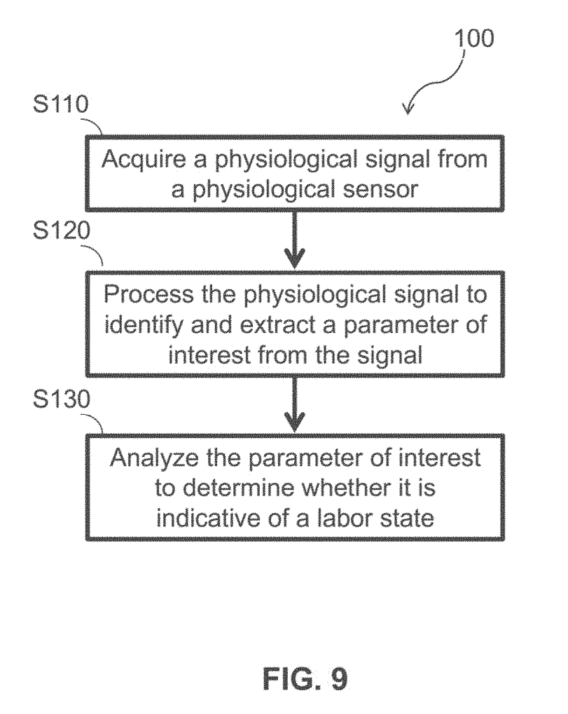

One non-limiting embodiment of a computer-implemented method 100 for identifying a labor state in a pregnant female is provided in FIG. 9. Such a method may be performed by any suitable device or system, such as, for example, any of the devices or systems described above.

As shown at block S110, the depicted method includes acquiring a physiological signal from a physiological sensor. The physiological signal may be one or more biopotential signals, for example, EHG, maternal ECG, and/or fetal ECG signals. In some embodiments, the physiological signal is acquired using a plurality of physiological sensors. In some embodiments, a plurality of physiological signals is acquired. For example, acquiring a physiological signal may include acquiring an EHG signal and, additionally or alternatively, one or more signals indicative of maternal uterine activity, maternal uterine muscle contractions, maternal heart electrical activity, maternal heart rate, fetal movement, fetal heart rate, maternal activity, maternal stress, and/or fetal stress. In various embodiments, the one or more physiological signals are sensed by a sensor having a plurality of electrodes and recorded by a processor into memory.

At block S120, the method includes processing the physiological signal to identify and extract a parameter of interest from the signal. The physiological signal may first undergo digital signal processing or signal processing via one or more signal processing components. The signal may be amplified, filtered, digitized, and/or otherwise processed to isolate a readable physiological signal from a noisy acquired signal. The physiological signal may undergo further processing by a computer processor to identify and extract a particular parameter of interest from the signal. The parameter of interest may be, for example, one or more of: a maternal heart rate metric, a maternal heart rate variability metric, a fetal heart rate metric, a fetal heart rate variability metric, a range of an electrohysterography signal, a power of an electrohysterography signal in a specific frequency band, a frequency feature of an electrohysterography signal, a time-frequency feature of an electrohysterography signal, a frequency of contractions, a duration of contractions, and an amplitude of contractions. In some embodiments, the metric (e.g., the maternal heart rate metric or fetal heart rate variability metric) is a mean value, a median value, a standard deviation, or any other meaningful statistic calculated from the signal. The parameter of interest may be a physiological parameter and/or a behavioral parameter. For examples, in some embodiments, the parameter of interest may be a measure of maternal anxiety or stress. In some embodiments, the parameter of interest may be an action, observed behavior, or feeling that is entered into the system by the pregnant woman or other user.

At block S130, the method includes analyzing the parameter of interest to determine whether the parameter is indicative of a labor state. Analyzing the parameter of interest is performed by a computer processor. In some embodiments, analyzing the parameter of interest includes comparing the parameter to community data stored in a database. In such embodiments, the systems and methods described herein may acquire signals and extract parameters of interest from a plurality of system users. For example, the systems and methods may be used by hundreds, thousands, hundreds of thousands, or millions of users, and the acquired physiological signals and/or extracted parameters of interest may be stored in a database. For example, for each user, the database may include physiological data along pregnancy, expected due date, actual baby's birth date, and notes associated with the data (e.g., times/dates when the user was in labor or times/dates when the user was experiencing false labor or Braxton Hicks contractions). The system or an administrator of the system may be able to identify or develop one or more trends, rules, correlations, and observations related to labor by tracking, aggregating, and analyzing the parameters from a plurality of users. For example, the data of a new user (i.e., a current user) may be compared with the data of all past users, to decide whether the new user is in labor state or non-labor state. In one embodiment, the data from the new user may be compared to the data from past users using, for example a two-class classification engine based on the data from all past users. In such embodiments, a classification engine may take the parameter(s) of interest as input, and assign a class to the parameter(s) of interest, for example a labor or non labor classification (i.e., a binary classifier). Alternatively, in some embodiments, the classification engine may assign a probability of belonging to a labor class to each of the parameter(s) of interest, and a probability of belonging to the non-labor class (i.e., Prob(non-labor)=1−Prob(labor)). Based on this probability, the system may provide a likelihood of being in labor to the new user.

As used herein, community data may refer to the plurality of stored physiological signals or extracted parameters and/or the trends, rules, correlations, observations, or other data derived from the signals and parameters.

Additionally or alternatively, in some embodiments, analyzing the parameter of interest includes feeding the parameter into a machine learning model or algorithm trained to detect labor. The machine learning model or algorithm may be trained to detect labor based on past physiological data and recorded experiences provided by past users of the system. The machine learning model may mine through vast quantities of data to identify common trends, rules, or correlations. The machine learning model may compare recorded data to observed outcomes to identify patterns that can be used to predict or identify labor. The machine learning model of some embodiments includes one or more of a generalized linear model, a decision tree, a support vector machine, a k-nearest neighbor, a neural network, a deep neural network, a random forest, and a hierarchical model. In other embodiments, any other suitable machine learning model may be used.

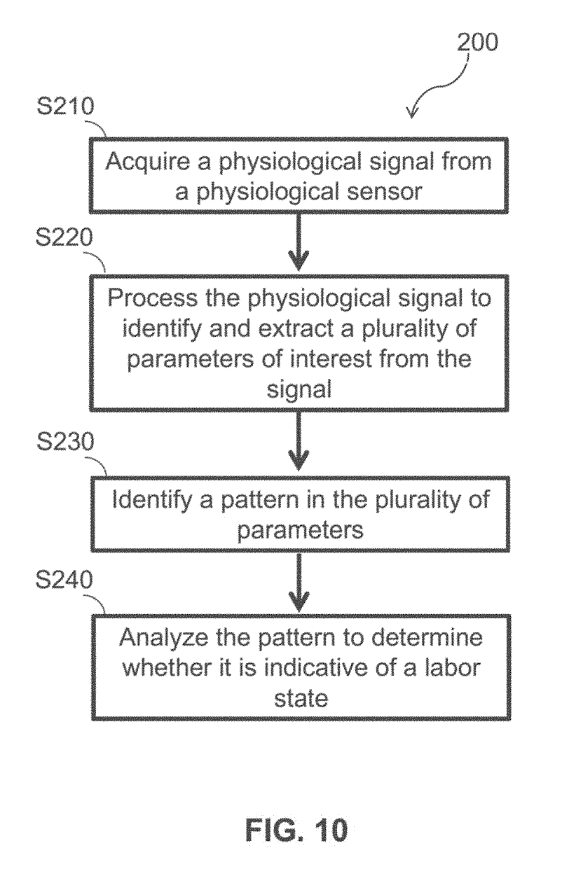

An additional embodiment of a computer-implemented method 200 for identifying a labor state in a pregnant female is provided in FIG. 10. As with the method 100 above, the method 200 of FIG. 10 includes: acquiring a physiological signal from a physiological sensor (S210), and processing the physiological signal to identify and extract a parameter of interest from the signal (S220). In the presently depicted method, a plurality of parameters is extracted. A plurality of parameters may be extracted from one physiological signal or one parameter each may be extracted from a plurality of physiological signals.

The method performed by a processor further includes identifying a pattern in the plurality of parameters (S230) and analyzing the pattern to determine whether the pattern is indicative of a labor state (S240). In some embodiments, block S240 is performed using simple decision trees, conditional logic, pattern recognition, or machine learning. Further, similar to the method 100 described above, in the present embodiment, patterns may be identified and characterized using community data stored in a database and/or machine learning models. Some non-limiting examples of patterns include: regular contractions, contractions increasing in intensity and frequency over time, periodic changes in maternal heart rate associated with contractions, periodic changes in belly shape or deformation (e.g., measured using an accelerometer), or decreased heart rate variability over time due to increased load on the autonomic nervous system of the user.

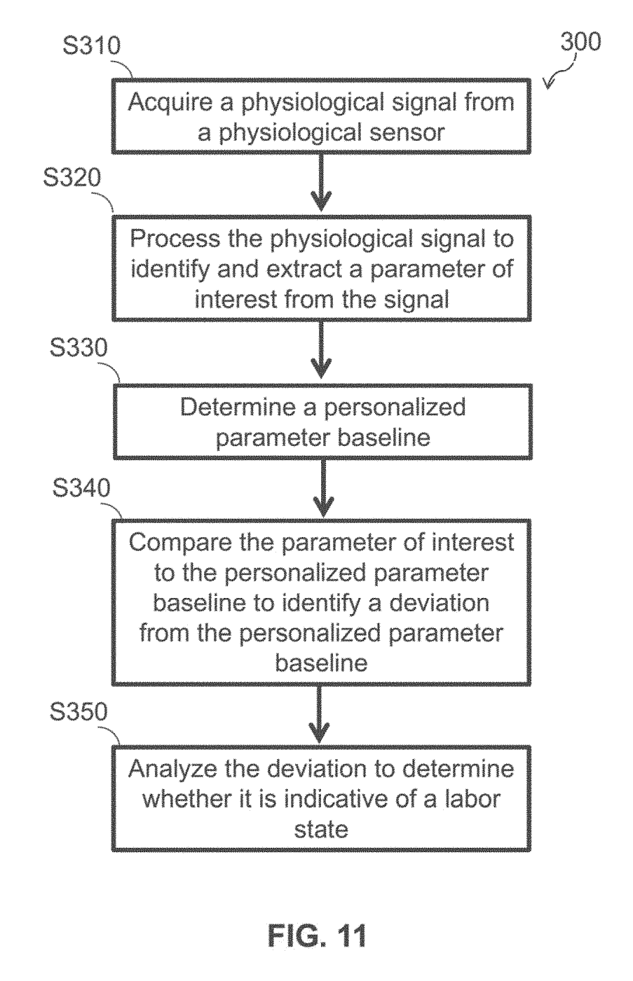

Another embodiment of a computer-implemented method 300 for identifying a labor state in a pregnant female is provided in FIG. 11. As with the above described methods, the method 300 of FIG. 11 includes: acquiring a physiological signal from a physiological sensor (S310), and processing the physiological signal to identify and extract a parameter of interest from the signal (S320). In the method 300 of FIG. 11, the processor additionally determines a personalized parameter baseline for the pregnant woman at block S330, compares the parameter of interest to the personalized parameter baseline to identify a deviation from the personalized parameter baseline at block S340, and analyzes the deviation to determine whether the deviation is indicative of a labor state at block S350. The personalized parameter baseline may be determined by tracking a parameter of interest over time and calculating a median value, an observed range of values, or other meaningful metric for that parameter. For example, in some embodiments, a personalized baseline may be calculated by taking a reference measurement during a calibration phase. In such embodiments, a calibration phase may occur, for example, the first time a user uses the device, at a pre-determined or stochastic interval (e.g., weekly), or before every recording. Alternatively, in some embodiments, a personalized baseline may be calculated by measuring one or more parameters of interest during specific and/or controlled conditions, for example, during sleep, during relaxation, during meditation, or during an activity in which the parameter of interest is stable, is relatively constant, or has a predictable pattern. Similar to the method 100 described above, in the present embodiment, deviations may be analyzed using community data stored in a database and/or machine learning models.

In some embodiments, a computer-implemented method for identifying a labor state in a pregnant female, such as any of the methods described above, also includes generating an alert related to the labor status. A command to generate the alert may be produced by the computer processor. The alert may be generated by a visual display, audio speakers, vibratory haptic feedback system, or other alert system located on the sensor module or mobile computing device. In some embodiments, the alert is a visual notification presented on a display screen providing an indication of labor status. In some embodiments, the alert is an auditory notification, such as an alarm, which sounds to provide an indication of labor status. In some embodiments, a vibration pattern may provide an indication of labor status.

The indication of labor status may include one or more of: a binary result (e.g., yes the woman is in labor or no the woman is not yet in labor), a probability that the woman is experiencing labor-inducing contractions, a degree of certainty around the determined probability, a probability that the pregnant female will enter the labor state within a given time period (e.g., within 12 hours, 24 hours, or 72 hours), and an estimate of time until the pregnant female enters the labor state. In some such embodiments, the method performed by the processor further includes calculating the relevant statistics, such as the probability that the woman is experiencing labor-inducing contractions, the degree of certainty around the determined probability, the probability that the pregnant female will enter the labor state within a given time period, and the estimate of time until the pregnant female enters the labor state.

In some embodiments, the computer-implemented method further includes sharing an alert related to the labor status with a contact. The alert may be sent automatically to one or more pre-selected contacts or pushed on demand when commanded by the pregnant woman user. For example, the alert may be shared with a gynecologist, obstetrician, other physician, nurse practitioner, veterinarian, other healthcare provider, doula, midwife, other birthing specialist, spouse, partner, parent, sibling, other family member, friend, a healthcare facility administrator, a service provider, or any other individual with whom the pregnant woman wishes to share such information. In some embodiments, upon detecting a positive labor status, the woman's healthcare provider and preferred healthcare facility are notified so that they may begin preparing for the woman's arrival. Alerts may be sent to contacts, for example, via an in-application notification, push notification, SMS text message, phone call, email, or any other suitable means of transmitting information.

In some embodiments, the computer-implemented method further includes sharing the acquired signal or the extracted parameters of interest with a contact such as a healthcare provider or birthing specialist for review.

In some embodiments, the method further includes performing an action based on the labor status. For example, in some embodiments, the method includes contacting a service provider to request services if the labor status is positive. Such services may include, but are not limited to, ride-sharing, taxi, childcare, pet-sitting, or other services a woman in labor may need to coordinate.

Unless otherwise defined, each technical or scientific term used herein has the same meaning as commonly understood by one of ordinary skill in the art to which this disclosure belongs.

As used in the description and claims, the singular form “a”, “an” and “the” include both singular and plural references unless the context clearly dictates otherwise. For example, the term “sensor” may include, and is contemplated to include, a plurality of sensors. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range, indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

The terms “connected” and “coupled” are used herein to describe a relationship between two elements. The term “connected” indicates that the two elements are physically and directly joined to each other. The term “coupled” indicates that the two elements are physically linked, either directly or through one or more elements positioned therebetween. “Electrically coupled” or “communicatively coupled” indicates that two elements are in wired or wireless communication with one another such that signals can be transmitted and received between the elements.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

The embodiments included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations and modifications of the above embodiments, and other embodiments not specifically described herein, will be apparent to those of skill in the art upon reviewing the above description. Thus, it should be understood that the invention generally, as well as the specific embodiments described herein, are not limited to the particular forms or methods disclosed, but also cover all modifications, equivalents, and alternatives falling within the scope of the appended claims.

1. A system for identifying a labor state in a pregnant female, the system comprising:
a patch coupled to an abdominal region of the pregnant female;
a physiological sensor coupled to the patch or integrated in the patch;
a processor communicatively coupled to the physiological sensor; and
a computer-readable medium having non-transitory, processor-executable instructions stored thereon, wherein execution of the instructions causes the processor to perform a method comprising:
acquiring a physiological signal from the physiological sensor;
processing the physiological signal to identify and extract a parameter of interest from the physiological signal; and
analyzing the parameter of interest to determine whether the parameter is indicative of a labor state.

2. The system of claim 1, wherein the method performed by the processor further comprises developing a personalized parameter baseline.

3. The system of claim 2, wherein the parameter of interest is tracked over time to develop the personalized parameter baseline.

4. The system of claim 2, wherein a plurality of parameters of interest are identified and extracted from the physiological signal, and
wherein analyzing the parameter of interest to determine whether the parameter is indicative of a labor state comprises: comparing the parameter of interest to the personalized parameter baseline to identify a deviation from the personalized parameter baseline, and determining whether the deviation is indicative of the labor state.

5. The system of claim 4, wherein analyzing the parameter of interest to determine whether the parameter is indicative of a labor state comprises: identifying a pattern in the plurality of parameters, and determining whether the pattern is indicative of the labor state.

6. The system of claim 4, wherein the plurality of parameters comprise physiological and behavioral parameters.

7. The system of claim 1, wherein analyzing the parameter of interest to determine whether the parameter is indicative of a labor state comprises feeding the parameter into a machine learning model trained to detect labor.

8. The system of claim 7, wherein the machine learning model comprises one or more of a generalized linear model, a decision tree, a support vector machine, a k-nearest neighbor, a neural network, a deep neural network, a random forest, and a hierarchical model.

9. The system of claim 1, wherein analyzing the parameter of interest to determine whether the parameter is indicative of the labor state comprises comparing the parameter to community data stored in a database.

10. The system of claim 9, wherein the community data comprises one or more of: recorded trends, rules, correlations, and observations generated from tracking, aggregating, and analyzing parameters from a plurality of users.

11. The system of claim 1, wherein the physiological sensor comprises a measurement electrode and reference electrode.

12. The system of claim 1, wherein the physiological sensor comprises one or more physiological sensors configured to measure one or more of an electrohysterography signal, a biopotential signal, maternal uterine activity, maternal uterine muscle contractions, maternal heart electrical activity, maternal heart rate, fetal movement, fetal heart rate, maternal activity, maternal stress, and fetal stress.

13. The system of claim 1, wherein the parameter of interest comprises one or more of a maternal heart rate metric, a maternal heart rate variability metric, a fetal heart rate metric, a fetal heart rate variability metric, a range of an electrohysterography signal, a power of an electrohysterography signal in a specific frequency band, a frequency feature of an electrohysterography signal, a time-frequency feature of an electrohysterography signal, a frequency of contractions, a duration of contractions, and an amplitude of contractions.

14. The system of claim 1, wherein the patch comprises a portable sensor module coupled to the patch or integrated into the patch, wherein the sensor module comprises the physiological sensor, the processor, and the computer-readable medium and further comprises an electronic circuit and a wireless antenna, and wherein the sensor module is in wireless communication with a mobile computing device.

15. The system of claim 1, wherein the method performed by the processor further comprises generating an alert.

16. The system of claim 1, wherein the method performed by the processor further comprises determining a probability that the pregnant female is experiencing labor-inducing contractions.

17. The system of claim 16, wherein the method performed by the processor further comprises determining a degree of certainty around the determined probability.

18. The system of claim 1, wherein the method performed by the processor further comprises determining a probability that the pregnant female will enter the labor state within a given time period.

19. The system of claim 1, wherein the method performed by the processor further comprises determining an estimate of time until the pregnant female enters the labor state.

20. A computer-implemented method for identifying a labor state in a pregnant female, the method comprising:
acquiring a physiological signal from a physiological sensor, wherein the physiological sensor is coupled to a patch or integrated into the patch, wherein the patch is configured to be coupled to an abdominal region of the pregnant female;
processing the physiological signal to identify and extract a parameter of interest from the physiological signal; and
analyzing the parameter of interest to determine whether the parameter is indicative of a labor state.
